(19) United States
(12) Patent Application Publication
(10) Pub. No.: US 2026/0314540 A1
(43) Pub. Date: Oct. 15, 2026

(54) SYSTEM AND METHOD OF PLANT GROWTH FREQUENCY STIMULATION

(71) Applicant: Graftan Darnall, MIAMI, FL (US)

(72) Inventor: Graftan Darnall, MIAMI, FL (US)

(21) Appl. No.: 19/176,147

(22) Filed: Apr. 11, 2025

(51) Int. Cl. A01G 7/04; G01N 33/00

(52) U.S. Cl. CPC A01G 7/04; G01N 33/0098

(57) ABSTRACT

The embodiments disclose a method of plant growth frequency stimulation including generating multiple frequencies at the same time for application on plants, synchronizing frequency adjustments with environmental factors including light cycles, nutrient schedules, and carbon dioxide enrichment for stimulating plant growth, broadcasting frequencies directed to plant seeds during stages of growth to stimulate plant growth, monitoring of plant health metrics with sensors in real-time, detecting and measuring environment conditions with a plurality of environment sensors, broadcasting frequencies directed to plants to drive away insect pests, and delivering automatically nutrients and carbon dioxide to seeds and plants.

### BACKGROUND

Plant growth varies by climate and seeding is performed to initiate growth. Typical plantings are simply dropping seed(s) in a shallow depression in soil or pots. Post planting water and some fertilizers are applied, and possible weeding is done. Yields vary on planting methods and post-planting care. The actual growth is left to nature.

### BRIEF DESCRIPTION OF DRAWINGS

Embodiments of the disclosure are illustrated by way of example and not by way of limitation in the figures of the accompanying drawings, in which like references indicate similar elements and in which:

FIG. 1 is a first exemplary embodiment of plant growth facilitation system.

FIG. 2 is an illustrative embodiment of the patent growth facilitation system activating one or more frequency generators to control acoustic transmissions represented by one or more types of sound waveforms being ultrasonic, audible, or infrasonic.

FIG. 3 is a second exemplary embodiment of plant growth facilitation system leveraging application control.

FIG. 4A illustrates an exemplary results in the use of acoustic transmissions on seeds deployed within a container of the plant growth facilitation system.

FIG. 4B illustrates an exemplary results in the use of acoustic transmissions on root growth associated with the germinating seed of FIG. 4A.

FIG. 4C illustrates an exemplary results in the use of acoustic transmissions on stem and leave growth associated with the plant produced by the seed of FIG. 4B.

FIG. 5 shows a block diagram of an overview of a flow chart of sensor-driven cultivation adjustments of one embodiment.

FIG. 6 shows a block diagram of an overview of a flow chart of gathering real-time sensor feedback of one embodiment.

FIG. 7 shows a block diagram of an overview of a flow chart of determining changes in plant responses of one embodiment.

FIG. 8 shows a block diagram of an overview of the flowering stage of one embodiment.

FIG. 9 shows a block diagram of an overview of the vegetative stage of one embodiment.

FIG. 10 shows a block diagram of an overview of the germination stage of one embodiment.

FIG. 11 shows a block diagram of an overview of pest control of one embodiment.

FIG. 12 shows for illustrative purposes only an example of a cultivation plant platform of one embodiment.

FIG. 13 shows a block diagram of an overview of a flow chart of soaking seeds for a predetermined time of one embodiment.

### DETAILED DESCRIPTION

Various embodiments of the disclosure are directed to plant growth facilitation system that is adapted to (i) control acoustic transmissions from emission sources that are configured to generate transmissions at different audio ranges inclusive of ultrasonic (>20 kHz), audible (≥20 Hz and ≤20 kHz), or infrasonic (<20 Hz) frequency ranges, (ii) conduct measurements of environmental conditions that influence plant growth through multiple sensors deployed throughout the plant growth facilitation system, and (iii) leverage artificial intelligence (AI) logic to conduct analytics of metrics captured by the sensors, compare these metrics against AI-based metrics, and dynamically alter the frequencies of the acoustic transmission(s) to optimize and automate operability of the plant growth facilitation system. According to one embodiment of the disclosure, these environmental sensors may be configured to detect light, temperature, humidity, carbon dioxide (CO₂) levels, and/or soil moisture, along with image capturing. A plurality of emission sources may generate acoustic transmissions ranging in frequency from between a few kilohertz (kHz) to over a hundred kilohertz such as a range of 0.5 kHz to 100 kHz.

### A. Terminology

In the following description, certain terminology is used to describe aspects of the invention. For example, in certain situations, the term “logic” is representative of hardware, firmware, or software that is configured to perform one or more functions. As hardware, logic may include circuitry having data processing or storage functionality. Examples of such circuitry may include, but are not limited or restricted to one or more hardware processors (e.g., a microprocessor with one or more processor cores, a digital signal processor, a programmable gate array, a microcontroller, an application specific integrated circuit “ASIC,” etc.), a semiconductor memory, or combinatorial elements. A portion of such circuitry may be deployed within a frequency generator or control system being part of the plant growth facilitation system.

Alternatively, logic (or module) may be software, such as executable code in the form of an executable application, a graphical user interface (GUI), an Application Programming Interface (API), a subroutine, a function, a procedure, an applet, a servlet, a routine, source code, object code, a shared library/dynamic library, or one or more instructions. The software may be stored in any type of a suitable non-transitory storage medium or transitory storage medium (e.g., electrical, optical, acoustical, or other forms of propagated signals such as carrier waves, infrared signals, or digital signals). Examples of the non-transitory storage medium may include, but are not limited or restricted to, a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device.

An “emission source” may be generally construed as logic with acoustic signal generation and transmission capabilities and/or a capability of connecting to any type of network, such as a public network (e.g., Internet), a private network (e.g., a wireless data telecommunication network, a local area network “LAN,” etc.), or a combination of networks. The emission source may include a speaker or other output logic to effectuate the sending of the acoustic transmission.

In certain instances, the terms “compare,” comparing,” “comparison,” or other tenses thereof generally mean determining if a match (e.g., identical or a prescribed level of correlation) is achieved between information under analysis.

A “message” generally refers to information transmitted in one or more electrical signals that collectively represent electrically stored data in a prescribed format. Each message may be in the form of one or more packets, frames, HTTP-based transmissions, or any other series of bits having the prescribed format. The message may initiate an acoustic transmission.

The character set “(s)” identifies one or more items. For example, the term “source(s)” constitutes one or more sources as the term “seed(s)” constitutes one or more seeds.

The term“ secondary metabolites” are compounds not directly involved in plant growth or reproduction but critical for plant defense, stress adaptation, and biochemical processes. For cannabis, these include: (1) cannabinoids (e.g., THC, CBD, CBG)—psychoactive and medicinal properties; (2) terpenes (e.g., myrcene, limonene, pinene)—flavor, aroma, and therapeutic effects; and/or (3) flavonoids (e.g., cannflavin A, B)—anti-inflammatory, antioxidant properties.

The term “florigen” (FT protein) is a plant hormone responsible for initiating and maintaining flowering in response to light cycles while “phytochrome” relates to a photoreceptor system that controls the transition between vegetative and flowering stages by detecting red (660 nm [nanometers]) and far-red (730 nm) light signals.

The term “ATP” or “adenosine triphosphate” constitutes the primary energy currency of the plant, which drives photosynthesis, nutrient absorption, and cellular growth. The term “enzymatic activation” is directed to the findings that certain acoustic frequencies enhance enzyme efficiency in ATP synthesis (via electron transport chain stimulation) and metabolic pathways.

The term “xylem” involves water Transport, namely that certain frequencies optimize capillary action, improving water uptake by a targeted plant. The term “phloem” involves sugar/metabolite transport, namely that acoustic waveforms influence sugar and secondary metabolite transport efficiency, ensuring optimized cannabinoid and terpene distribution.

With respect to light frequency synergy, acoustic waveform frequencies interact with light absorption, improving photosynthetic rates. In fact, specific frequencies enhance stomatal conductivity, increasing CO₂ uptake efficiency and modulated acoustic waveforms increase chlorophyll production, improving light-energy conversion efficiency.

Lastly, the terms “or” and “and/or” as used herein are to be interpreted as inclusive or meaning any one or any combination. Therefore, “A, B, or C” or “A, B, and/or C” mean “any of the following: A; B; C; A and B; A and C; B and C; A, B, and C.” An exception to this definition will occur only when a combination of elements, functions, steps, or acts are in some way inherently mutually exclusive.

### B. General Architecture

It should be noted that the descriptions that follow, for example, in terms of a method of plant growth frequency stimulation are described for illustrative purposes and the underlying system can apply to any number and multiple types of plants. In one embodiment of the disclosure, the method of plant growth frequency stimulation provided by the plant growth facilitation system can be configured using automated nutrient and CO₂ delivery systems. The plant growth facilitation system can be configured to include a control system, one or more frequency generators, emission sources that may include speakers and/or emitters, and/or sensors communicatively coupled to the control system.

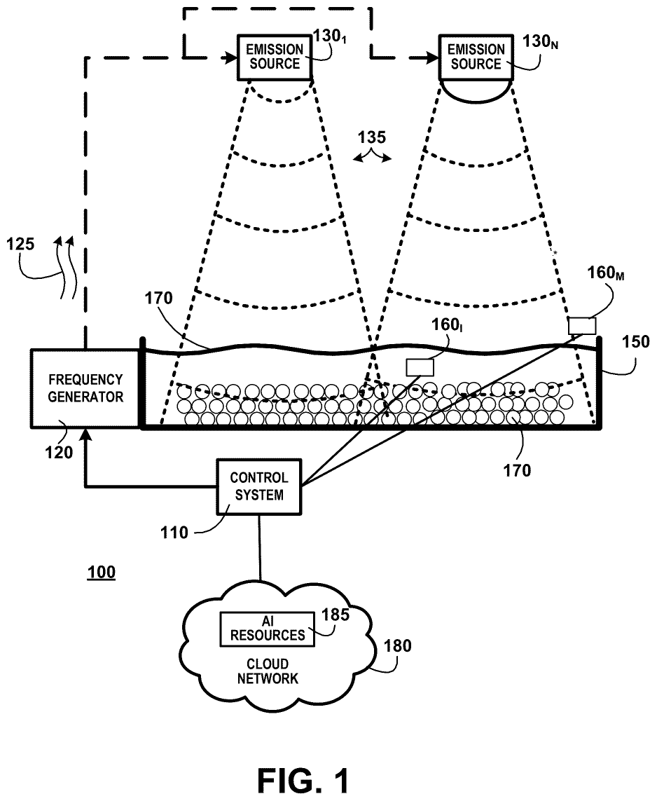

Referring to FIG. 1, a first exemplary embodiment of plant growth facilitation system 100 is shown. Herein, the plant growth facilitation system 100 features a control system 110, one or more frequency generators 120 (hereinafter, “frequency generator(s) 120”), one or more sound emission source(s) 130₁-130_{N} (N≥1), optional light source(s) 140, cultivation platform 150, and a plurality of environmental sensors 160₁-160_{M} (M≥1). The plant growth facilitation system 100 is configured to deliver acoustic transmissions of specified frequencies to seeds 170 within the cultivation platform 150 such as a germination soak container. Stated differently, the emission source(s) 130₁-130_{N} provide acoustic transmissions (e.g., frequency-based sound waveforms) 135 with tailored frequencies to facilitate growth by seeds or plants positioned in the cultivation platform 150 as well as facilitate pest and disease suppression or resistance.

More specifically, a control system 110 may be communicatively coupled to a cloud network 180 with Artificial Intelligence (AI) resources 185 adapted to operate in a training mode to receive training data associated with metrics that have been or could be captured by environmental sensors in response to acoustic transmission of a prescribed frequency or acoustic transmissions of prescribed frequencies. These metrics signify targeted measurements that signify a physical environment that is conducive to favorable to plant growth and/or favorable pest or plant disease suppression or resistance. The AI resources 185 may include large language models (LLMs), which are adapted to identify patterns of acoustic and/or light transmission(s) that are advantageous for plant growth and provide information associated with such acoustic and/or light patterns to the control system 110. The information associated with such patterns is provided from the AI resources 185 to the control system 110, which dynamically controls the frequency generator(s) 120 based on the AI-resource information to effectively control the operability of the emission source(s) 130₁-130_{N}.

For example, the control system 110 may be configured to instruct the frequency generator(s) 120 to transmit adjusted frequency control signals 125 to at least one emission source(s) 130₁-130_{N}. In response to the frequency control signals 125, the emission sources (e.g., speakers) direct the acoustic transmissions 135 to the cultivation platform 150. The cultivation platform 150, in this example, may be deployed as a germination soak container that contains seeds 170 (e.g., hemp seeds) soaking in water. This germination process creates micropores in the seeds 170 to accelerate the absorption of water and liquid nutrients leading to faster growth. The control system 110 application of sound frequencies tailored to germination range from 0.5 kHz to 1.5 kHz of one embodiment.

Referring still to FIG. 1, based on the acoustic transmissions 135 that is capable of producing a multi-frequency band of acoustic waveforms (e.g., series of ultrasonic, audible, and/or infrasonic waveforms) directed to the seeds 170, a seedling and germination process is commenced by the plant growth facilitation system 100 to begin cultivation. For one embodiment, a process conducted by the control system 110 performs a dynamic frequency output using at least an emission source such as a computing device that emits sound, ultrasound, and/or infrasound to improve and accelerate the growth of plants produced from the seeds 170. The process is adaptable for use in growing hemp plants as well as other types of plants. In this example, during seedling and germination growth stages, the frequencies emitted from the emission source(s) 130 range from 0.1 kHz to 10 kHz.

To provide feedback to the control system 110, which may be shared with the AI resources for training, the plant growth facilitation system 100 incorporates environmental sensors 160₁-160_{M} to continuously monitor the environmental conditions that affect the plants. It is contemplated that some of the environmental sensors 160₁-160_{M} may be further configured to detect light cycles, which impact the growth rates of the hemp seeds 170 and/or plants. It is contemplated that acoustic waveform simulation may be synchronized with light waveform of a selected wavelength or wavelength range to enhance different processes of the targeted plants during dark and light cycles. Also, dynamic frequency modulation during dark cycles may enhance phytochrome reset efficiency, improving flowering among other things.

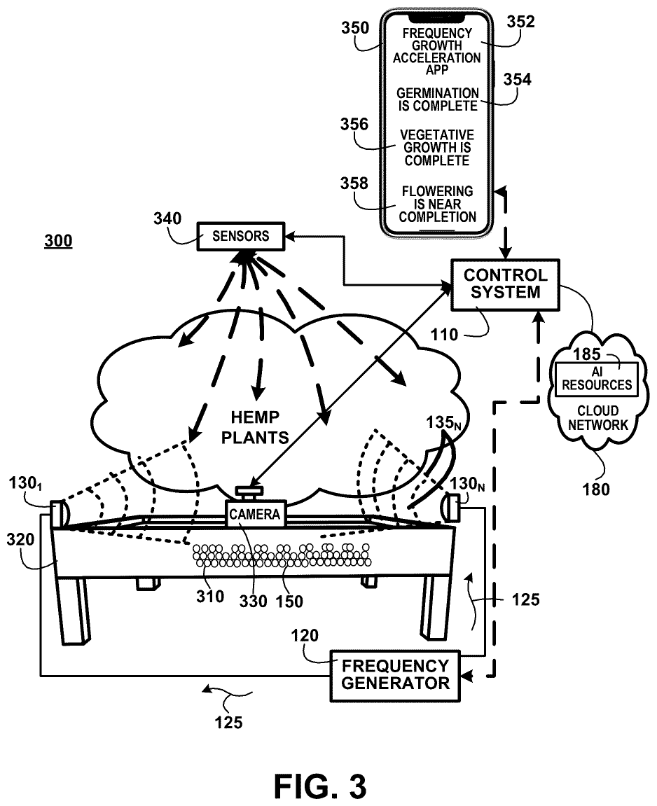

It is further contemplated that some of the environmental sensors 160₁-160_{M} may be further configured to measure, within a physical environment associated with the cultivation platform 150, the following: (i) ambient air temperature, (ii) air humidity, (iii) CO₂ levels, and/or (iv) water temperature within the cultivation platform 150. If soil is used to grow the plants, the soil moisture may be measured by an environmental sensor in lieu of the water temperature. Also, one of the environmental sensors 160₁-160_{M} (see sensor 330 of FIG. 3) may be an image capturing device (e.g., camera, CCD, etc.) to capture images of plants produced from the seeds 170, which may be provided to the AI resources to determine whether plant growth is proceeding as expected and the overall health of the plant (e.g., along with the presence of any pests. The images may be analyzed for plant size and overall health of the plant (e.g., stomatal activity, stock density/thickness, flower bud size, chlorophyll content, etc.).

The metrics provided from the environmental sensors 160₁-160_{M} are analyzed, and the results of the analyses may cause the control system 110 (based on assistance from the AI resources 185 as needed) to dynamically adjust the signaling to the frequency generator(s) 120 and thereby cause adjustments in the frequency control signals 125 to alter the frequency waveforms produced by the emission source(s) 130₁-130_{N}. For example, the frequencies associated with the acoustic transmissions 135 from the emission source(s) 130₁-130_{N} may differ during daylight and nighttime, during different seasons, during different local weather patterns. Stated differently, the acoustic (and light) frequency variations may be adjusted dynamically based on environmental factors and desired results to maintain a steady growth rate during impactful light cycles of one embodiment.

According to one embodiment of the disclosure, the plant growth facilitation system 100 may be configured to operate to enhance germination of hemp seeds and the cultivation of hemp plants through the application of acoustic frequencies. The system generates and transmits specific frequency ranges at different stages of a plant's lifecycle and at different times in the day to stimulate plant growth, flowering, and resin during the plant's lifecycle, as well as the integration with environmental sensors and AI resources as feedback and analytic processes to ensure that optimal plant growth is achieved for the given environment.

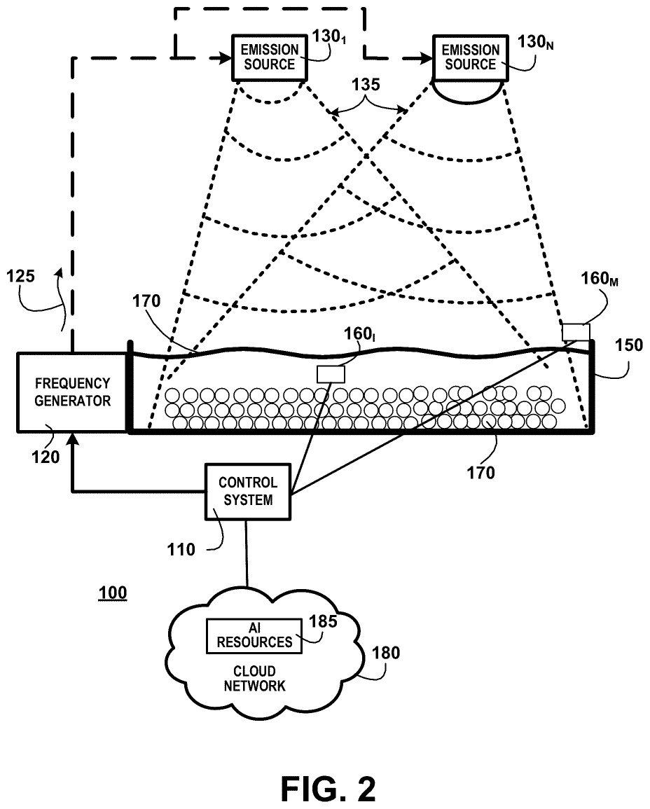

Referring to FIG. 2, an illustrative embodiment of the patent growth facilitation system activating the frequency generator(s) 120 to control the emission source(s) to generate the acoustic transmissions 135, which may be represented by acoustic transmissions of different frequencies such as a first acoustic transmission 135₁ up to a N^{th} acoustic transmission 135_{N}. Each of these emission source(s) 130₁-130_{N} may be configured to emit acoustic transmissions 135₁-135_{N} as sound waveforms, where the frequency ( [symbol] ) of the sound waveforms 135₁-135_{N} may be set and vary between different selective frequency bands, such as an ultrasonic frequency band (e.g. [symbol] >20 kHz), an audible frequency band (20 KHz≥ [symbol] ≥20 Hz), or infrasonic frequency band ( [symbol] <20 Hz). It is contemplated that the frequency bands may be of a smaller frequency range, such as a few kilohertz (e.g., 2-5 kHz).

Alternatively, each of these emission source(s) 130₁-130_{N} may be configured to emit sound waveforms with a static frequency band such as a first emission source 130₁ may be configured to emit sound waveforms 135₁ with an audible frequency band while a second emission source 130_{N} may be configured to emit sound waveforms 135_{N} with an ultrasonic frequency band or an infrasonic frequency band.

Where each of the emission source(s) 130₁-130_{N} is configured to emit sound waveforms of different frequency bands, the lateral or radial coverage area of each sound waveform 135₁-135_{N} provided by the emission source(s) 135₁-135_{N} may overlap each other so that sound waveform frequencies for the different emission source(s) 135₁-135_{N} may be sequenced to provide sufficient amounts of sounds waveforms of different frequency bands. This deployment may be utilized when frequency changes to facilitate plant ground is to be conducted in a serial fashion. However, to support concurrent, multi-frequency band transmissions, where sound waveforms of different frequency bands are provided from the emission source(s) 130₁-130_{N} to the seeds 170 concurrently (i.e., overlapping at least partially in time), the coverage area by each of the emission source(s) 130₁-130_{N} should reach all of the seeds 170. Stated differently, where the emission source(s) 130₁-130_{N} support emission of sound waveforms at specific, different frequency bands, the seeds 170 within the cultivation platform 150 may need to be positioned to receive sound waveforms of different frequency bands from the different emission sources (e.g., each seed is adapted to receive sound waveforms from both emission sources 130₁ and 135_{N}). These frequency bands may constitute ultrasonic, audible, and/or or infrasonic frequency bands or prescribed frequency ranges that are found to assist in plant growth.

Referring now to FIG. 3, a second exemplary embodiment of plant growth facilitation system 300 is shown, where the system 300 is adapted to leveraging application control. Herein, the control system 110 is configured to instruct the frequency generator(s) 120 to transmit adjusted frequency acoustic transmissions 135₁-135₂ from the emission source(s) 130₁-130_{N} such as speakers 130₁-130₂. The speakers 130₁-130₂ direct the frequency waveforms to hemp seeds and/or plants 310 in a cultivation platform 320. The cultivation platform 320 has at least one imaging sensor 330 (e.g., camera) mounted to provide images of the hemp seeds/plants 310 and any insect pests present. The images are analyzed by the processor (in control system 110) to determine the plant growth progress. The processed images of insect pests are used to determine the type of insect to adjust frequencies known to drive away that type of insect. The processed images of plant growth are used to determine the stage of growth of the plants to adjust frequencies known to optimize growth at any particular stage of growth of one embodiment.

A user mobile device 350 having a frequency growth acceleration application 352 displays, on a graphical user interface of the user mobile device 350, the status of the plant growth. For example, the status may identify that the germination stage has been completed 354, the vegetative growth stage has been complete 356, and the flowering stage is near completion 358. The sensors 340 are placed in numerous locations in proximity to the plants to detect and measure plant conditions of one embodiment. The output from the sensors 340 (e.g., parameters extracted from measured data such temperature, humidity/moisture, light, etc.) and/or output from the camera 330 (e.g., parameters extracted from captured images) may be provided to the control system 110 for processing and processed results provided to an application on the user mobile device 350. This allows a user, remotely located from the cultivation platform 320, to monitor and finely tune plant growth by allowing the user to make adjustments to the frequency control signals 125 provided to the emission source(s) 130. Hence, the user is able to alter sound waveform generation that the AI resources 185 within the cloud network 180 have identified as more conducive for plant generation.

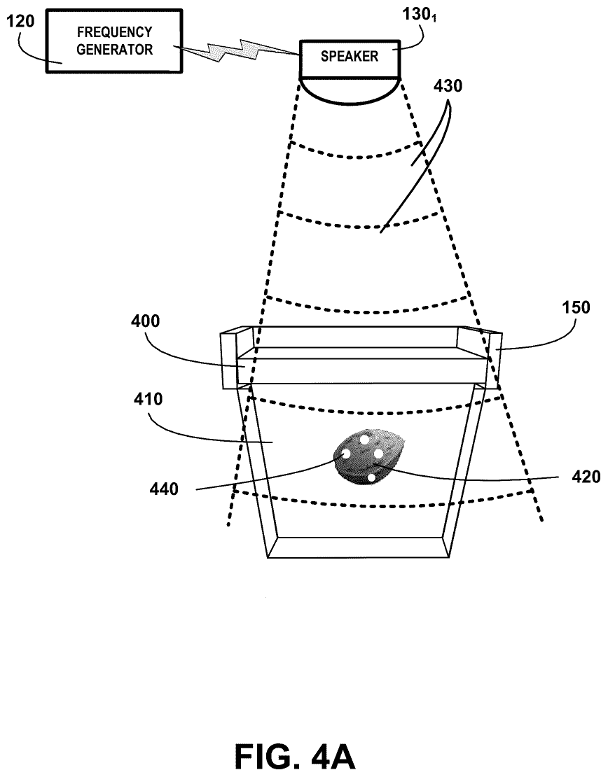

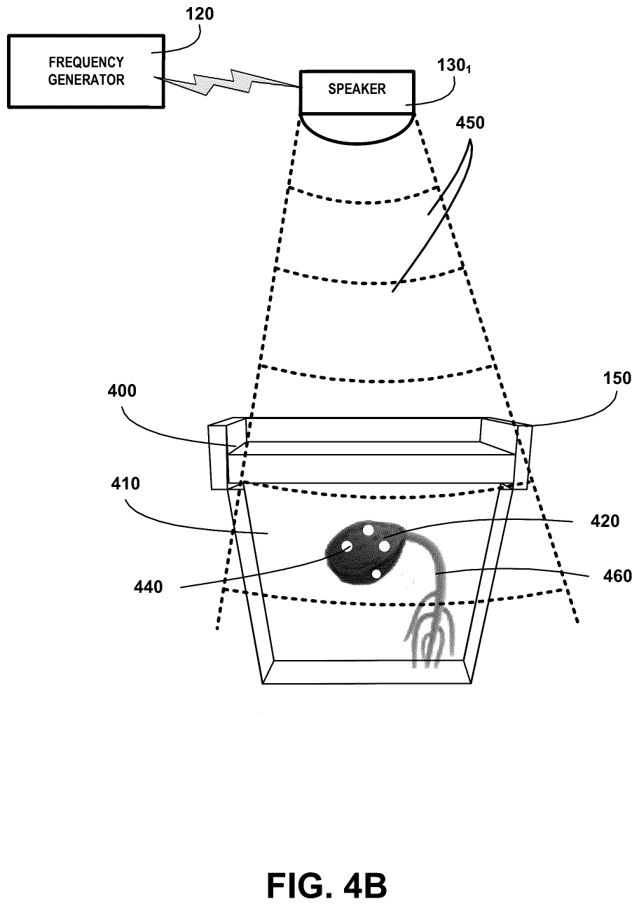

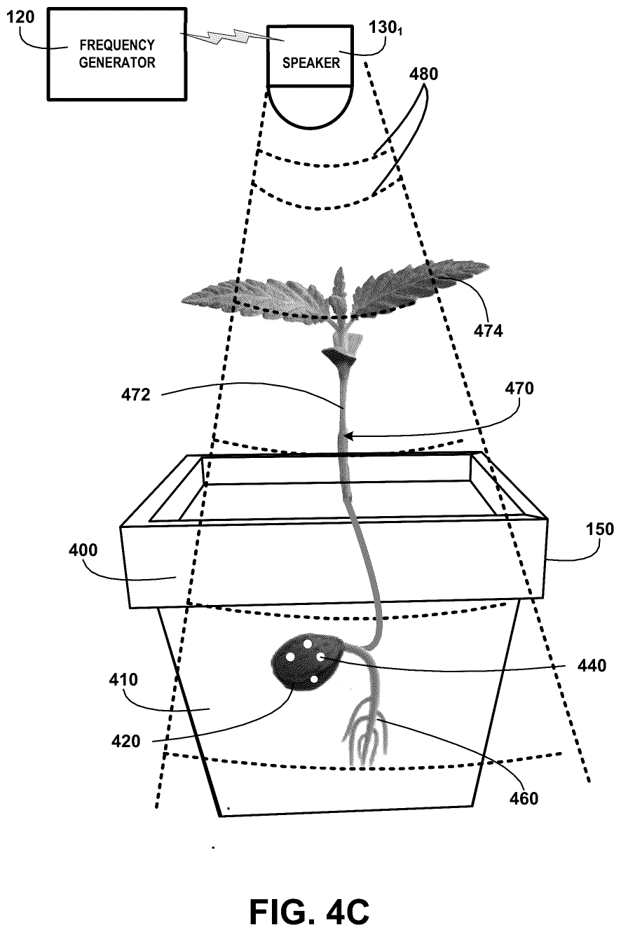

Referring to FIG. 4A, an exemplary results in the use of acoustic transmissions 135₁ from the emission source 130₁ on seeds 170 deployed within the cultivation platform 150 of the plant growth facilitation system is shown. As shown, the cultivation platform 150 is filled with planting media 400 containing planting media nutrients 410. A hemp plant seed 420 is placed in the planting media 400. Micro-pores 440 created by frequency signal(s) 430 within the acoustic transmission 135₁, such as sound waveforms of different bands (e.g., audible, ultrasonic, infrasonic) are transmitted concurrently or separately, where the selected frequency or frequencies have been determined by AI resources 185 to encourage faster absorption of water and liquid nutrients by the hemp plant seed 420. The frequency generator(s) 120 wirelessly (or through a wired interconnect) transmits frequencies to at least one emission source(s), such as speaker 130₁ for example, to broadcast the frequency signal(s) 430 to the hemp plant seed 420. The frequency signal(s) 430 stimulate growth in the hemp plant seed 420 of one embodiment.

Referring now to FIG. 4B, an exemplary results in the use of acoustic transmissions on root growth associated with the germinating hemp plant seed 420 of FIG. 4A is shown. Herein, the cultivation platform 150 represented as a single plant container filled with the planting media 400 containing planting media nutrients 410. A hemp plant seed 420 is placed in the planting media 400. Micro-pores 440 created by frequency signals 450, which may be different sound waveform frequencies and/or a different pattern of sound waveform frequencies, are seen that allow faster absorption of water and liquid nutrients. The frequency generator(s) 120 is wirelessly (or through a wired interconnect) transmitting frequencies to at least one speaker 130₁ to broadcast frequency signals 450 to the hemp plant seed 420. The stimulation of the growth produced by the use of sonic (1 Hz-3 kHz) and ultrasonic (20 kHz-30 kHz) frequencies to enhance growth, flowering, and resin production in hemp plants. In this example, root growth 460 is shown from the hemp plant seed 420 of one embodiment.

Referring to FIG. 4C, an exemplary results in the use of acoustic transmissions 135₁ on stem and leave growth associated with the plant 470 produced by the seed 420 of FIGS. 4A-4B is shown. Herein, the cultivation platform 150 represented as the single plant container filled with the planting media 400 containing planting media nutrients 410. A hemp plant seed 420 is placed in the planting media 400. Micro-pores 440 created by frequency signals 480, which may be different sound waveform frequencies and/or a different pattern of sound waveform frequencies than other growth cycles of FIGS. 4A-4B, are seen that allow faster absorption of water and liquid nutrients. The frequency generator(s) 120 is wirelessly transmitting frequencies to at least one speaker 130₁ to broadcast frequency signals 480 to the hemp plant seed 420.

The micropores created by the frequency signals 480 are seen that allow faster absorption of water and liquid nutrients by the seed of one embodiment.

The stimulation of the growth extends to the root growth 460, stem growth 472, and leaves growth 474. The system integrates adaptive frequency modulation with environmental control systems, including light cycles, nutrient delivery, and CO₂ enrichment, to maximize yield, cannabinoid production, and terpene synthesis. The flowering growth begins as the stem growth 472 and the leaves growth 474 vegetative growth may be stimulated by a frequency ranging from 1.5-3.0 kHz and reaches the completion of one embodiment.

The flowering stages and secondary metabolite production in the hemp plants are stimulated by the application of frequencies for flowering ranging with a prescribed frequency range, such as 2.0 kHz to 4.5 kHz, and late resin production stages ranging from 3.0 kHz to 5.0 kHz. Secondary metabolites are compounds not directly involved in plant growth or reproduction but critical for plant defense, stress adaptation, and biochemical processes. In cannabis, these include: (1) cannabinoids, (2) terpenes, and/or (3) flavonoids. Stated differently, results associated with the application of selected acoustic waveforms to secondary metabolites activate mild stress responses by the targeted plant (e.g., cannabis), boosting trichome density and increasing cannabinoid and terpene biosynthesis.

The application of these frequencies is increasing flower size and density, enhancing resin output and trichome density, and improving terpene profiles and cannabinoid concentrations. The systems are generating in the cultivation area one or more frequencies at the same time of one embodiment. For example, light and frequency synchronization may be conducted with the integration of specific frequencies with light cycles, including photoperiod-sensitive stages (e.g., 12/12 light cycles). Use of frequencies during both light and dark cycles to enhance flowering hormone production and optimize the photosynthetic efficiency of one embodiment.

Secondary metabolite production is stimulated with frequencies to stimulate cannabinoid and terpene biosynthesis by activating specific metabolic pathways. Pairing frequencies with nutrient delivery for phosphorus (P), sulfur (S), and potassium (K) during resin and trichome production. Adaptive environmental control by integration of sensors for real-time monitoring of plant health metrics (e.g., stomatal activity, chlorophyll content) of one embodiment.

### C. General Methods of Operation

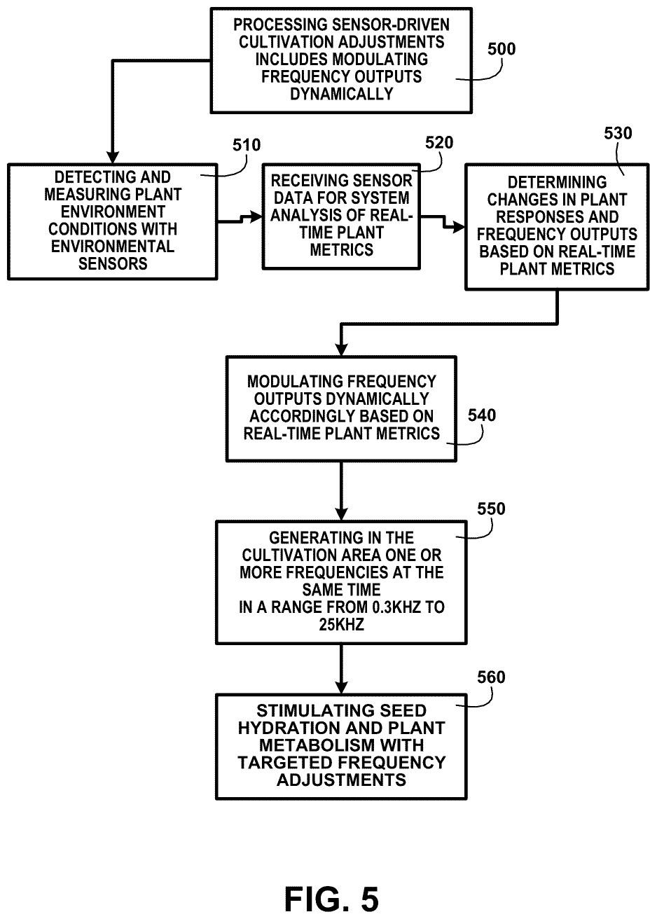

Referring to FIG. 5, a block diagram of an overview of a flow chart of sensor-driven cultivation adjustments of one embodiment. Herein, the processing sensor-driven cultivation adjustments includes modulating frequency outputs dynamically based on several conditions (block 500). The sensors detect and measure plant environment conditions with environmental sensors (block 510). The sensors measure plant internal conditions as well as external observable plant changes. Receiving sensor data for system analysis of real-time plant metrics (block 520) is transmitted with wireless sensors.

In one embodiment of the disclosure, the machine learning and AI-driven systems analyze the sensor data for determining changes in plant responses and frequency outputs based on real-time plant metrics (block 530). The determinations utilize comparisons of plant responses under similar conditions and the resulting plant responses. The comparisons may adjust modulating frequency outputs dynamically frequencies accordingly based on real-time plant metrics (block 540) as compared to previous results of one embodiment.

The system in one embodiment generates in the cultivation area one or more frequencies at the same time in a range from 0.3 kHz to 120 kHz (block 550). The dynamic adjustments are stimulating seed hydration and plant metabolism with targeted frequency adjustments (block 560). The system comprises a frequency generator, speakers, and environmental sensors to deliver tailored frequencies to plants of one embodiment.

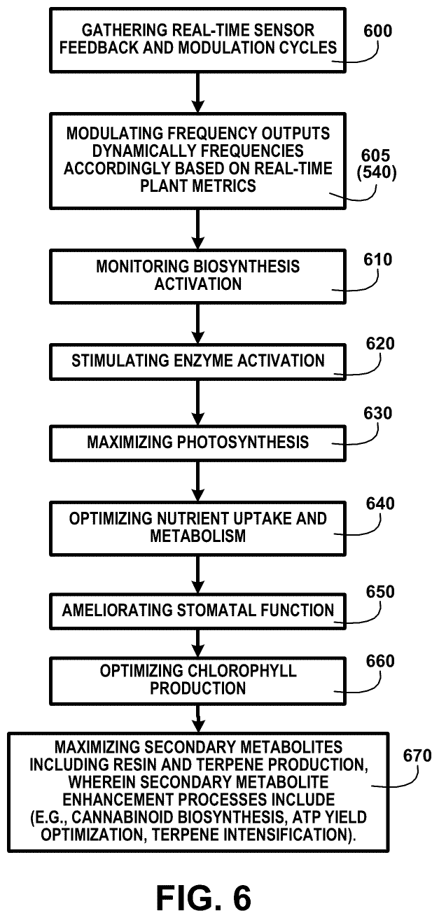

Referring now to FIG. 6, a block diagram of an overview of a flow chart of gathering real-time sensor feedback is shown. Herein, a process for gathering real-time sensor feedback and modulation cycles is conducted (block 600). The systems are modulating frequency outputs dynamically accordingly based on real-time plant metrics 440 using current sensor data that include monitoring biosynthesis activation (block 610). The dynamic frequencies adjustment is stimulating enzyme activation (block 620), maximizing photosynthesis (block 630), optimizing nutrient uptake and metabolism (block 640), ameliorating stomatal function (block 650), optimizing chlorophyll production (block 660), and maximizing secondary metabolites including resin and terpene production (block 670) of one embodiment.

These are natural plant functions that are kept at optimal levels with the application of the frequencies dynamic responses to changing environmental conditions, in one embodiment. In this embodiment, the system integration of adaptive frequency modulation with environmental control systems, including light cycles, nutrient delivery, and CO₂ enrichment, maximizes yield, cannabinoid production, and terpene synthesis to improve the quality of the plant products of one embodiment.

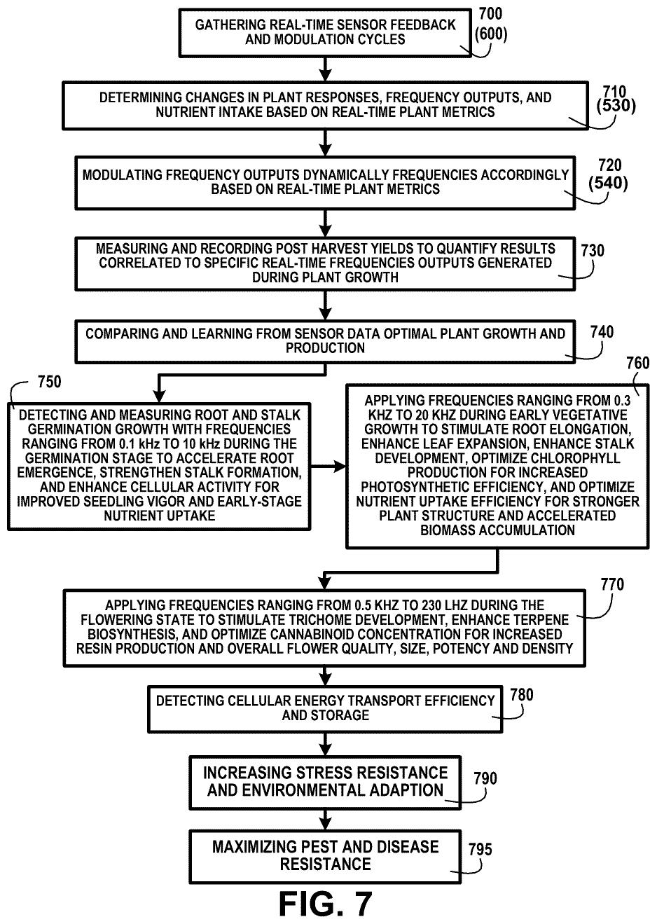

Referring to FIG. 7, a block diagram of an overview of a flow chart of determining changes in plant responses of one embodiment. Herein, a process for gathering real-time sensor feedback and modulation cycles is conducted (block 700, 600) in order to gather of real-time sensor feedback and modulation cycles, determine changes in plant responses and frequency outputs based on real-time plant metrics (block 710, 530), and the modulating frequency outputs dynamically frequencies accordingly based on real-time plant metrics (block 720, 540) of one embodiment.

The sensors measuring and recording post-harvest yields to quantify results correlated to specific real-time frequencies outputs generated during plant growth (block 730) provide a firm basis for determining frequency modulation over the growth cycles and real-time plant responses. The data is used for comparing and learning from sensor data optimal plant growth and production (block 740). The growth stages sensor data includes detecting and measuring root and stalk germination growth with frequencies ranging from 0.1 kHz to 10 kHz (block 750). As the plant matures the shift is for detecting and measuring stem and leaf vegetative development with frequencies ranging from 0.3 kHz to 20 kHz (block 760) of one embodiment.

The final growth stage includes detecting and measuring flowering development with frequencies ranging from 0.1 kHz to 120 kHz for trichome enhancement and terpene intensification 632. At each stage detecting cellular energy transport efficiency and storage (block 770), increasing stress resistance and environmental adaption (block 780), and maximizing pest and disease resistance increase yields and plant product qualities of one embodiment (block 790).

Adaptive environmental control includes the integration of sensors for real-time monitoring of plant health metrics (e.g., stomatal activity, and chlorophyll content). AI-driven adjustments of frequencies, light, and nutrient schedules based on sensor feedback aid plant growth. Sensors monitoring environmental conditions and plant responses allow automated nutrient and CO₂ delivery systems synchronized with sound treatments of one embodiment.

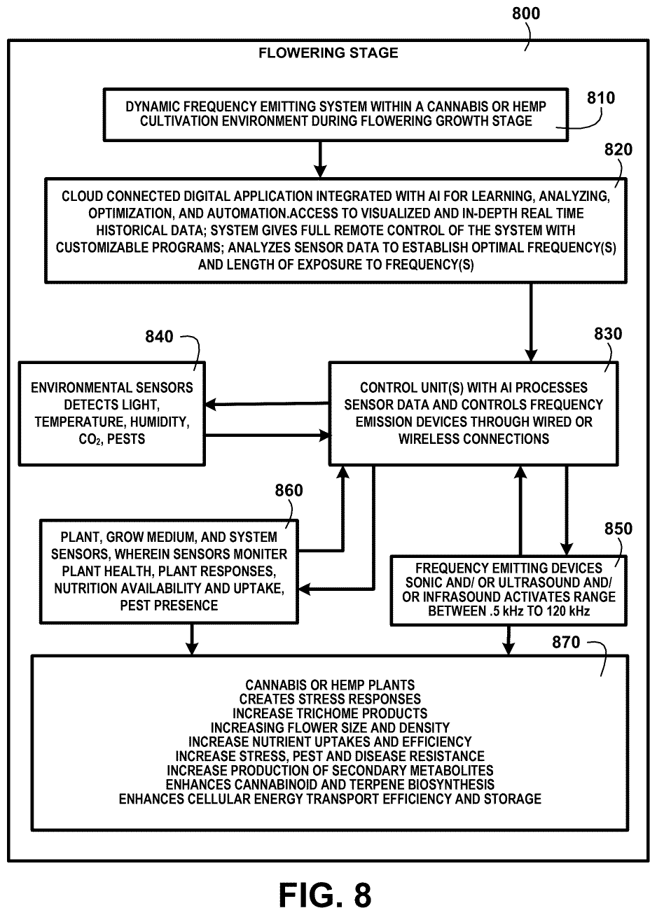

Referring to FIG. 8, a block diagram of an overview of the flowering stage of one embodiment is shown. Herein, a flowering stage 800 with a dynamic frequency emitting system within a cannabis or hemp cultivation environment during flowering growth stage block 810). A cloud-connected digital application integrated with AI for learning, analyzing, optimization, automation, and access to visualized and in-depth real-time historical data, wherein the system gives full remote control of the system with customizable programs; analyzes sensor data to establish optimal frequency(s) and length of exposure to frequency(s) (block 820). A plurality of control unit(s) with AI processes sensor data and controls frequency emission devices through wired or wireless connections (block 830).

Environmental sensors detect light, temperature, humidity, CO₂, and pests (block 840). Frequency emitting devices sonic and/or ultrasound and/or infrasound activates in a range between 0.5 kHz to 120 kHz (block 850). As also shown in FIG. 8, a plant, grow medium, and system sensors, wherein sensors monitor plant health, plant responses, nutrition availability and uptake, and pest presence (block 860). The system impacts cannabis or hemp plants and creates stress responses, increases trichome products, increases flower size and density, increases nutrient uptakes and efficiency, increases stress, pest and disease resistance, increases production of secondary metabolites, enhances cannabinoid and terpene biosynthesis, enhances cellular energy transport efficiency and storage (block 870) of one embodiment.

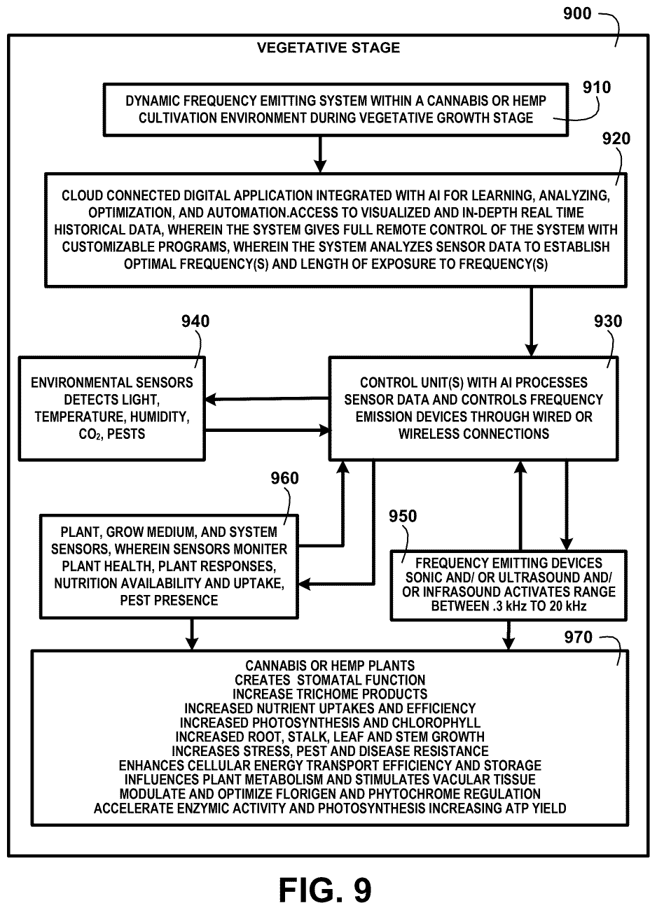

Referring now to FIG. 9, a block diagram of an overview of the vegetative stage 900 of one embodiment is shown. Herein, a vegetative stage with a dynamic frequency emitting system within a cannabis or hemp cultivation environment during vegetative growth stage (block 910) is shown. A cloud-connected digital application integrated with AI for learning, analyzing, optimization, automation, and access to visualized and in-depth real-time historical data, wherein the system gives full remote control of the system with customizable programs, wherein the system analyzes sensor data to establish optimal frequency(s) and length of exposure to frequency(s) (block 920). A plurality of control unit(s) with AI, processes sensor data and controls frequency emission devices through wired or wireless connections (block 930). Environmental sensors detect light, temperature, humidity, CO₂, and pests (block 940). Frequency emitting devices sonic and/or ultrasound and/or infrasound activates range between 0.3 kHz to 20 kHz (block 950).

Referring still to FIG. 9, a plant, grow medium, and system sensors are operational, wherein sensors monitor plant health, plant responses, nutrition availability and uptake, and pest presence (block 960). The system impacts cannabis or hemp plants and creates stomatal function; increases trichome products; increases nutrient uptakes and efficiency; increases photosynthesis and chlorophyll; increases root, stalk, leaf and stem growth; increases stress, pest and disease resistance; enhances cellular energy transport efficiency and storage; influences plant metabolism and stimulates vascular tissue; modulate and optimize florigen and phytochrome regulation; and accelerate enzymic activity and photosynthesis increasing ATP yield (block 970) of one embodiment.

Hence, the results associated with the application of selected acoustic waveforms to florigen and phytochrome regulation include a synchronization of acoustic waveforms with photoperiod cues, optimizing florigen signaling and phytochrome conversion efficiency. Also, AI-driven adaptive frequencies may be selected and dynamically adjust based on detected light cycles, ensuring proper floral transition timing. Also, acoustic waveforms of particular frequencies may be selected to enhance electron transport in mitochondria, leading to higher ATP output and enzymatic activation for metabolic reactions.

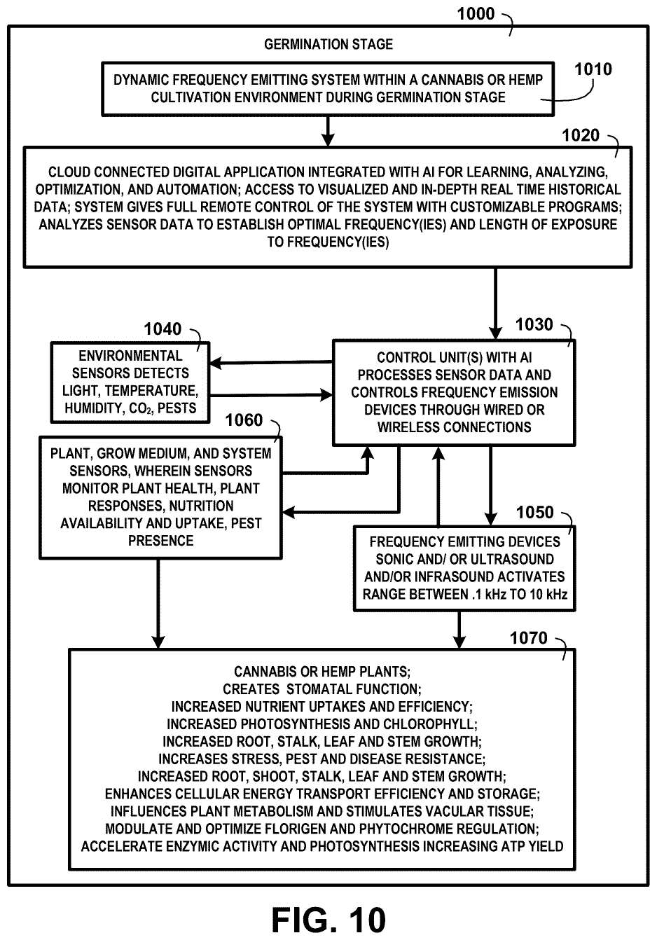

Referring to FIG. 10, a block diagram of an exemplary overview of the germination stage 1000 is shown. Herein, the germination stage 1000 with a dynamic frequency emitting system within a cannabis or hemp cultivation environment during germination stage (block 1010). A cloud-connected digital application integrated with AI for learning, analyzing, optimization, automation, and access to visualized and in-depth real-time historical data. The system gives full remote control of the system with customizable programs and analyzes sensor data to establish optimal frequency(ies) and length of exposure to frequency(ies) (block 1020). Control unit(s) with AI process sensor data and control frequency emission devices through wired or wireless connections (block 1030). Environmental sensors detect light, temperature, humidity, CO₂, and pests (block 1040). Frequency emitting devices sonic and/or ultrasound and/or infrasound activates in a range between 0.1 kHz to 10 kHz (block 1050).

Referring still to FIG. 10, a plant, grow medium, and system sensors are in operation, wherein sensors monitor plant health, plant responses, nutrition availability and uptake, and pest presence (block 1060). The system impact on cannabis or hemp plants creates stomatal function; increased nutrient uptakes and efficiency; increased photosynthesis and chlorophyll; increased root, stalk, leaf and stem growth; increases stress, pest and disease resistance; increased root, shoot, stalk, leaf and stem growth; enhances cellular energy transport efficiency and storage; influences plant metabolism and stimulates vascular tissue; modulate and optimize florigen and phytochrome regulation; and accelerate enzymic activity and photosynthesis increasing ATP yield (block 1070) of one embodiment.

In summary, for vascular tissue transport and nutrient uptake, the acoustic waveforms may be used to increase xylem fluid movement, optimizing water and nutrient uptake and/or, as acoustic pulses, stimulate phloem flow, ensuring better distribution of nutrients.

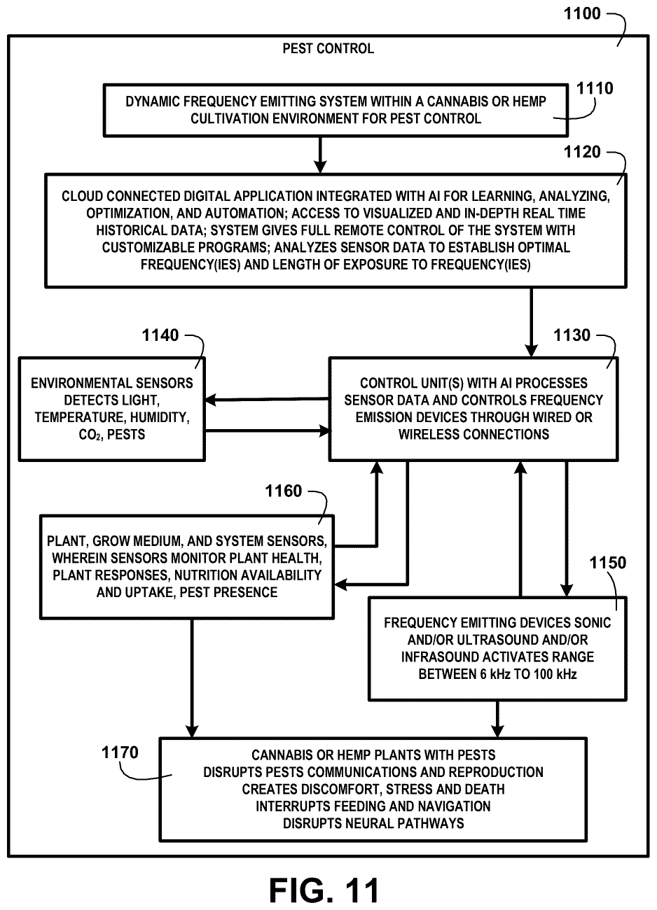

Referring to FIG. 11, a block diagram of an exemplary overview of pest control 1100 is shown. Herein, pest control with a dynamic frequency emitting system within a cannabis or hemp cultivation environment for pest control is conducted (block 1110). A cloud-connected digital application integrated with AI for learning, analyzing, optimization, automation, and access to visualized and in-depth real-time historical data. The system gives full remote control of the system with customizable programs and analyzes sensor data to establish optimal frequency(ies) and length of exposure to frequency(ies) (block 1120). The system includes a control unit(s) with AI that processes sensor data and controls frequency emission devices through wired or wireless connections (block 1130).

Environmental sensors detect light, temperature, humidity, CO₂, and pests (block 1140). A plurality of frequency emitting devices sonic and/or ultrasound and/or infrasound activates in a range between 6 kHz to 100 kHz (block 1150). As still shown in FIG. 11, a plant, grow medium, and system sensors are in operation, wherein sensors monitor plant health, plant responses, nutrition availability and uptake, and pest presence (block 1160) with cannabis or hemp plants with pests. The emitted optimal frequency(ies) and length of exposure to frequency(ies) disrupts pests' communications and reproduction, creates discomfort, stress, and death, interrupts feeding and navigation, and disrupts neural pathways (block 1170) of one embodiment.

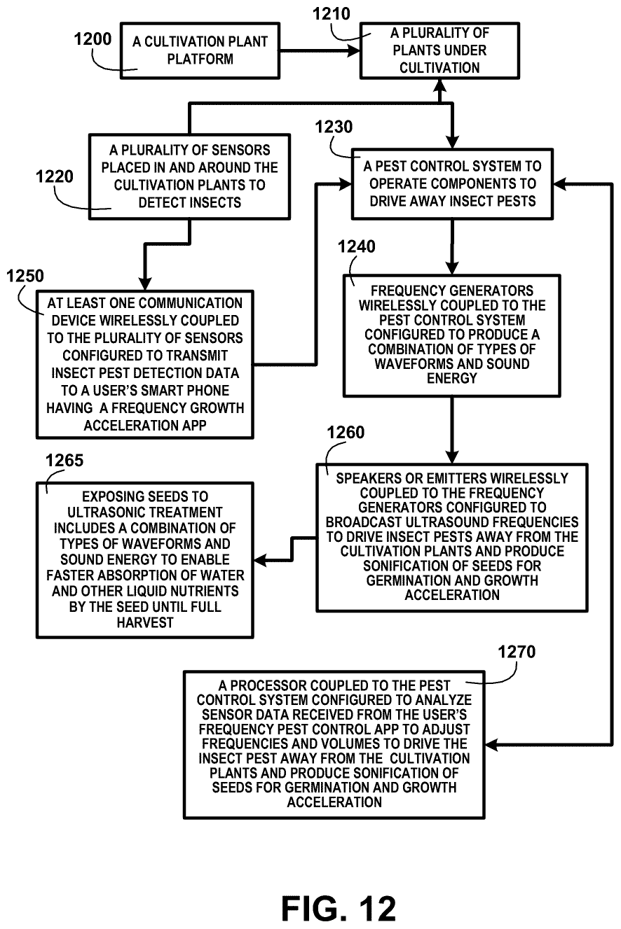

Referring to FIG. 12, an example of an operational flow 1200 associated with the cultivation platform 150 of FIGS. 1-3 is shown. Herein, the cultivation platform 150 supporting a plurality of plants under cultivation (block 1210). A plurality of sensors are placed in and around the cultivation plants to detect insects (block 1220). A pest control system to operate components to drive away insect pests (block 1230) further improves plant health and growth optimization. Frequency generators wirelessly coupled to the pest control system are configured to produce a combination of types of waveforms and sound energy (block 1240). At least one communication device wirelessly coupled to the plurality of sensors configured to transmit insect pest detection data to a user's smartphone having a frequency growth acceleration app (block 1250) of one embodiment.

Exposing seeds to ultrasonic treatment includes a combination of types of waveforms and sound energy to enable faster absorption of water and other liquid nutrients by the seed until full harvest (block 1255). Speakers or emitters wirelessly coupled to the frequency generators configured to broadcast ultrasound frequencies to drive insect pests away from the cultivation plants and produce sonification of seeds for germination and growth acceleration (block 1260). A processor coupled to the pest control system configured to analyze sensor data received from the user's frequency pest control app to adjust frequencies and volumes to drive the insect pest away from the cultivation plants and produce sonification of seeds for germination and growth acceleration (block 1270) as shown in this embodiment.

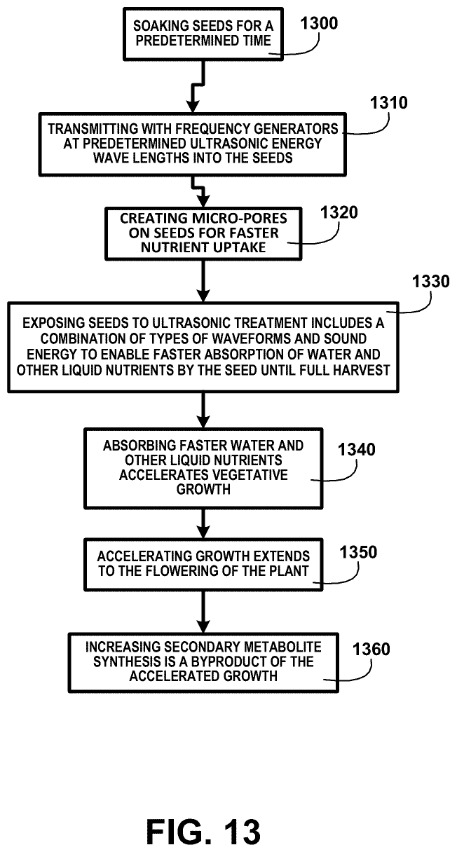

Referring now to FIG. 13, a block diagram of an exemplary overview of a flow chart of soaking seeds for a predetermined time is shown. Herein, the seeds are soaked for a predetermined time for germination (block 1300). Transmitting with frequency generators at predetermined ultrasonic energy wavelengths into the seeds (block 1310) for creating micro-pores on seeds for faster nutrient uptake (block 1320).

Exposing seeds to ultrasonic treatment includes a combination of types of waveforms and sound energy to enable faster absorption of water and other liquid nutrients by the seed until full harvest (block 1330). The micro-pores created on seeds enable absorbing faster water and other liquid nutrients accelerating vegetative growth (block 1340). Accelerating growth extends to the flowering of the plant (block 1350). Increasing secondary metabolite synthesis is a byproduct of the accelerated growth (block 1360) of one embodiment.

The use of sonic (1 Hz-3 kHz) and ultrasonic (20 kHz-30 kHz) frequencies to enhance growth, flowering, and resin production in cannabis and hemp plants. Application of frequencies for germination, vegetative growth, flowering, and secondary metabolite synthesis of one embodiment.

In one embodiment of the present invention, AI-driven adjustments of frequencies, light, and nutrient schedules are used based on sensor feedback. Embodiment of the disclosure describe a system that includes a modular hardware including one or more frequency generators emitting sound waves of a precise acoustic frequency or a range of acoustic frequencies, speakers and emitters, sensors monitoring environmental conditions and plant responses, automated nutrient and carbon-dioxide (CO₂) delivery systems synchronized with sound treatments, cameras, sensors for monitoring temperature, light, frequency, water and nutrient absorption measurements, databases, artificial intelligence, machine learning, wireless digital communication devices, frequency growth acceleration app, mobile devices, user graphical interface, processors, and others of one embodiment.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

1. A method of plant growth frequency stimulation for a cannabis or hemp plant, comprising:
generating, with one or more emission sources, acoustic transmissions at a plurality of frequency bands;
broadcasting acoustic transmissions including a first acoustic transmission of a first frequency band during a first growth stage of the plant, a second acoustic transmission of a second frequency band during a second growth stage of the plant, and a third acoustic transmission of a third frequency band during a third growth stage of the plant, wherein the first frequency band, the second frequency band, and the third frequency band are different from each other;
synchronizing the acoustic transmissions having different frequency bands with different frequency adjustments with environmental factors including light cycles, nutrient schedules, or carbon dioxide enrichment for stimulating plant growth;
broadcasting frequencies directed to plant seeds during stages of growth to stimulate plant growth based on sensor-driven cultivation adjustments; and
monitoring, in real-time and using one or more sensors directed at the plant for measuring plant health metrics with sensors in real-time; and
dynamically adjusting at least one of the frequency or duration of the broadcasting based at least on at least one of (i) the measured plant health metrics or (ii) one or more environmental conditions including a current light cycle.

2. The method of claim 1 further comprising:
detecting and measuring environment conditions with a plurality of environment sensors;
broadcasting frequencies directed to plants to drive away insect pests; and
automatically delivering nutrients and carbon dioxide (CO₂) to seeds and plants.

3. The method of claim 1, wherein the first growth stage corresponds to a germination stage, the second growth stage corresponds to a vegetative stage, and the third growth stage corresponds to a flowering stage.

4. The method of claim 1, wherein the broadcasting of the frequencies directed to plant seeds during stages of growth comprises generating frequencies ranging from 0.1 kHz to 10 kHz during germination, broadcasting of the frequencies directed to plant seeds during stages of growth comprises generating frequencies ranging from 0.3 kHz to 20 kHz during the vegetative stage and generating frequencies ranging from 0.5 kHz to 120 kHz during the flowering stage.

5. The method of claim 1, wherein at least a first light cycle operating at a first selected wavelength range is synchronized with the broadcasting of the second acoustic transmission and a second light cycle operating at a second selected wavelength range different from the first selected wavelength range is synchronized with the broadcasting of the third acoustic transmission.

6. The method of claim 1, wherein the broadcasting of the acoustic transmissions including the first acoustic transmission of the first frequency band comprises analyzing and comparing plant frequency responses with AI-driven systems to adjust sound frequencies dynamically for optimal growth.

7. The method of claim 1, wherein the broadcasting of the acoustic transmissions comprises pairing frequencies with nutrient delivery for phosphorus (P), sulfur(S), and potassium (K) during resin and trichome production.

8. The method of claim 1, wherein the broadcasting of the acoustic transmissions comprises applying acoustic transmissions at the first frequency band for a germination stage, applying acoustic transmissions at the second frequency band for the vegetative stage, and applying acoustic transmissions at the third frequency band for the flowering stage.

9. The method of claim 8, wherein the first frequency band is different from the second frequency band or the third frequency band.

10. The method of claim 8, wherein the first frequency band is different from the second frequency band and the third frequency band, and the second frequency band is different from the third frequency band.

11. A method of plant growth frequency stimulation for a cannabis or hemp plant, comprising:
generating multi-frequency acoustic transmissions with one or more emission sources;
broadcasting the multi-frequency acoustic transmissions at a first frequency band during a germination stage of the plant, at a second frequency band during a vegetative stage of the plant, and at a third frequency band during a flowering stage of the plant, the first, second, and third frequency bands being different from one another, and transitioning between the first, second, and third frequency bands in synchronization with a transition in a light cycle of a cultivation environment containing the plant to plant seeds or plants during different stages of growth to stimulate plant growth;
monitoring of plant health metrics with sensors in real-time.
monitoring, in real-time and using one or more sensors directed at the plant for measuring physiological characteristics of the plant, plant health metrics; and
within a current growth stage, dynamically modulating frequencies of one or more sound waveforms of the multi-frequency acoustic transmissions based on at least one of (i) the plant health metrics or (ii) one or more environmental conditions including the light cycle.

12. The method of claim 11, wherein the generating of the multi-frequency acoustic transmissions includes including generating a first acoustic transmission of a first frequency band during a first growth stage of the plant, generating a second acoustic transmission of a second frequency band during a second growth stage of the plant, and generating a third acoustic transmission of a third frequency band during a third growth stage of the plant, wherein the first frequency band, the second frequency band, and the third frequency band are different from each other.

13. The method of claim 11, wherein the first growth stage corresponds to a germination stage, the second growth stage corresponds to a vegetative stage, and the third growth stage corresponds to a flowering stage.

14. The method of claim 13, wherein at least a first light cycle operating at a first selected wavelength range is synchronized with the broadcasting of the second acoustic transmission and a second light cycle operating at a second selected wavelength range different from the first selected wavelength range is synchronized with the broadcasting of the third acoustic transmission.

15. (canceled)

16. The method of claim 11 further comprising:
detecting and measuring environment conditions with a plurality of environment sensors; and
broadcasting frequencies directed to plants to drive away insect pests, or
automatically delivering nutrients and carbon dioxide (CO₂) to seeds and plants.

17. A method of plant growth frequency stimulation, comprising:
generating, with one or more emission sources, acoustic transmissions at a plurality of frequency bands
broadcasting acoustic transmissions including a first acoustic transmission of a first frequency band during a first growth stage of the plant, a second acoustic transmission of a second frequency band during a second growth stage of the plant, and a third acoustic transmission of a third frequency band during a third growth stage of the plant, wherein the first frequency band, the second frequency band, and the third frequency band are different from each other;
synchronizing the first acoustic transmission with a first light cycle, the second acoustic transmission with a second light cycle and a third acoustic transmission with a third light cycle, where the first light cycle is different than the second light cycle and different from the third light cycle;
broadcasting frequencies directed to plant seeds during stages of growth to stimulate plant growth based on sensor-driven cultivation adjustments; and
monitoring, in real-time and using one or more sensors directed at the plant for measuring plant health metrics with sensors in real-time; and
dynamically adjusting at least one of the frequency or duration of the broadcasting based at least on at least one of (i) the measured plant health metrics or (ii) one or more environmental conditions including a current light cycle.

18. The method of claim 17, wherein the first growth stage corresponds to a germination stage, the second growth stage corresponds to a vegetative stage, and the third growth stage corresponds to a flowering stage.

19. The method of claim 17, further comprising analyzing and comparing plant frequency responses with AI-driven systems to adjust sound frequencies dynamically for optimal growth.

20. The method of claim 17, further comprising pairing frequencies with nutrient delivery for phosphorus (P), sulfur (S), and potassium (K) during resin and trichome production.
